(19) United States
(12) Patent Application Publication
(10) Pub. No.: US 2022/0142760 A1
(43) Pub. Date: May 12, 2022

(54) ORAL CARE APPLIANCE WITH LIGHT ILLUMINATION FOR TEETH WHITENING AND GUM HEALTH

(71) Applicant: KONINKLIJKE PHILIPS N.V., EINDHOVEN (NL)

(72) Inventor: TIANYI WANG, KENMORE, WA (US)

(21) Appl. No.: 17/436,688

(22) PCT Filed: Feb. 14, 2020
(86) PCT No.: PCT/EP2020/053951

§ 371 Date: Sep. 7, 2021

Related U.S. Application Data
(60) Provisional application No. 62/815,454, filed on Mar. 8, 2019.

(51) Int. Cl. A61C 19/06; A61N 5/06

(52) U.S. Cl. CPC A61C 19/066; A61N 5/0613; A61N 2005/063; A61N 2005/0606; A61N 5/0603

(57) ABSTRACT

An oral appliance having a body, the body including a plate member and a lighting system. The plate member having a first surface, the first surface including a top portion, a center portion, and a bottom portion. The lighting system includes a first lighting sub-system having at least one first light source, where the at least one first light source arranged in the center portion of the plate member, and a second lighting sub-system having at least one second light source, where the at least one second light source arranged in the top portion of the plate member and/or the bottom portion of the plate member. The at least one first light source is arranged to produce a first light at a first wavelength and the at least one second light source is arranged to produce a second light at a second wavelength.

### FIELD OF THE DISCLOSURE

The present disclosure is directed generally to oral care appliances, more specifically to systems and methods for whitening teeth and increasing overall gum health.

### BACKGROUND

Staining and yellowing of teeth is an inevitable result of daily use. As a result, there has been an increase in consumer products that are designed to provide at-home treatments for teeth whitening. One type of at-home product emits targeted light in the “blue” wavelengths of the electromagnetic spectrum. Blue light can be used in conjunction with hydrogen peroxide (H₂O₂) to facilitate the reaction between hydrogen peroxide and staining molecules to increase the effectiveness of teeth whitening.

Additionally, light therapy or phototherapy, is a known technology that has been used by healthcare professionals to relieve pain, heal wounds, fight infections, rejuvenate aging skin, treat acne, and to improve conditions such as seasonal affective disorder (SAD). Phototherapy involves delivery of low-level “red” light at specific wavelengths to improve blood flow, increase circulation, encourage cellular turnover, and repair damaged tissue. Note that while light therapy typically involves delivery of “red” light wavelengths, and are referred to herein as “red” light, other light wavelengths, including those outside of the red or infrared spectrums, can be used, depending on the treatments to be executed, and the light wavelengths that are most effective for those treatments.

Notwithstanding available teeth whitening treatments, there is a continued need for improved systems and methods for whitening teeth and increasing overall gum health simultaneously.

### SUMMARY OF THE DISCLOSURE

The instant disclosure is directed to an oral care appliance that produces a first wavelength of light directed to a user's teeth to facilitate teeth whitening, while simultaneously producing a second wavelength of light directed to a user's gums to promote overall gum health, providing a more complete oral healthcare within the same treatment time.

Generally, according to an aspect of the disclosure, there is provided an inventive oral care appliance having a body, the body including a plate member and a lighting system. The plate member having a first surface, the first surface including a top portion, a center portion, and a bottom portion. The lighting system includes a first lighting sub-system having at least one first light source, where the at least one first light source is arranged in the center portion of the plate member, and a second lighting sub-system having at least one second light source, where the at least one second light source is arranged in the top portion of the plate member and/or the bottom portion of the plate member. The at least one first light source is arranged to produce a first light at a first wavelength and the at least one second light source is arranged to produce a second light at a second wavelength.

According to an aspect, the body further includes a bite member having a bite surface arranged to contact a user's teeth.

According to an aspect, the first surface of the plate member is a curved surface.

According to an aspect, the first wavelength is in the range of 400-495 nm.

According to an aspect, the second wavelength is in the range of 650-1400 nm.

According to an aspect, the at least one first light source is separated from the at least one second light source by a first vertical distance.

According to an aspect, at least one of the at least one first light source and the at least one second light source is a light-emitting diode (LED).

According to an aspect, the at least one first light source and the at least one second light source are selected from the group of: a light diffusing fiber (e.g., single mode, multimode, or photonic crystal fibers), an electrodeless light source, an incandescent light source, a florescent light source, a hollow-cathode light source, a sulfur light source, a neon and argon light source, a laser light source, and a plasma light source.

According to another aspect, an oral care appliance is provided, the oral care appliance includes a body, the body includes a plate member and a lighting system. The plate member includes a first surface, the first surface having a top portion, a center portion, and a bottom portion. The lighting system includes a first lighting sub-system having at least one first light source, where the at least one first light source is arranged in the center portion of the plate member, and a second lighting sub-system having at least one second light source and at least one third light source, where the at least one second light source is arranged in the top portion of the plate member and the at least one third light source is arranged in the bottom portion of the plate member. The at least one first light source is arranged to produce a first light at a first wavelength, and the at least one second light source is arranged to produce a second light at a second wavelength.

According to another aspect, the body further includes a bite member having a bite surface arranged to contact a user's teeth.

According to another aspect, the first surface of the plate member is a curved surface.

According to another aspect, the first wavelength is in the range of 400-495 nm and the second wavelength is in the range of 650-1400 nm.

According to another aspect, the at least one first light source is separated from the at least one second light source by a first vertical distance (D1).

According to another aspect, at least one of the at least one first light source and the at least one second light source is a light-emitting diode (LED).

According to another aspect, the at least one first light source, the at least one second light source, and the at least one third light source are selected from the group of: a light diffusing fiber (e.g., single mode, multimode, or photonic crystal fibers), an electrodeless light source, an incandescent light source, a florescent light source, a hollow-cathode light source, a sulfur light source, a neon and argon light source, a laser light source, and a plasma light source.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a perspective view of an oral care appliance according to the present disclosure.

FIG. 2 is a top perspective view of an example embodiment of a plate member according to the present disclosure.

FIG. 3 is a top perspective view of an example embodiment of a plate member according to the present disclosure.

FIG. 4 is a top plan view of an oral care appliance according to the present disclosure.

FIG. 5 is a cross-sectional view of the first and second wavelengths of light directed to a user's tooth and gums.

### DETAILED DESCRIPTION OF EMBODIMENTS

The present disclosure describes various embodiments of an oral appliance for whitening teeth and increasing overall gum health. The oral care appliance has a body including a plate member and a lighting system. The plate member has a first surface, the first surface includes a top portion, a center portion, and a bottom portion. The lighting system includes a first lighting sub-system having at least one first light source, where the at least one first light source is arranged in the center portion of the plate member, and a second lighting sub-system having at least one second light source, where the at least one second light source is arranged in the top portion of the plate member and/or the bottom portion of the plate member. The at least one first light source is arranged to produce a first light at a first wavelength and the at least one second light source is arranged to produce a second light at a second wavelength.

This arrangement produces an improved overall oral healthcare experience in that the first light (e.g., “blue” light) is more efficiently focused on the user's teeth for more efficient whitening, and the second light (e.g., “near infrared” or “NIR” light) is more efficiently focused on the user's gums for more efficient absorption by the gums improving overall gum health. The specific wavelengths of light for the first and second lights do not have to be specifically blue and red or near-infrared; any wavelengths suitable to the specific treatment outcome desired can be used. These two light applications can happen simultaneously, allowing for two separate treatments within a single treatment time-frame. Furthermore, the physical separation between these light sources (e.g., D1 and/or D2 discussed in detail below), provides for a more effective and more uniform/homogenous illumination by the first and second lights on the teeth and gums, respectively. Additionally, physical separation of these sources of light may aid in heat dispersion, i.e., allowing for an enhanced control of thermal exposure to the teeth, and more importantly, to the user's gums. Furthermore, the separation allows for lower power requirements for the first and second light sources due to each source being closer to the area it is designed to effect, e.g., the teeth or the gums.

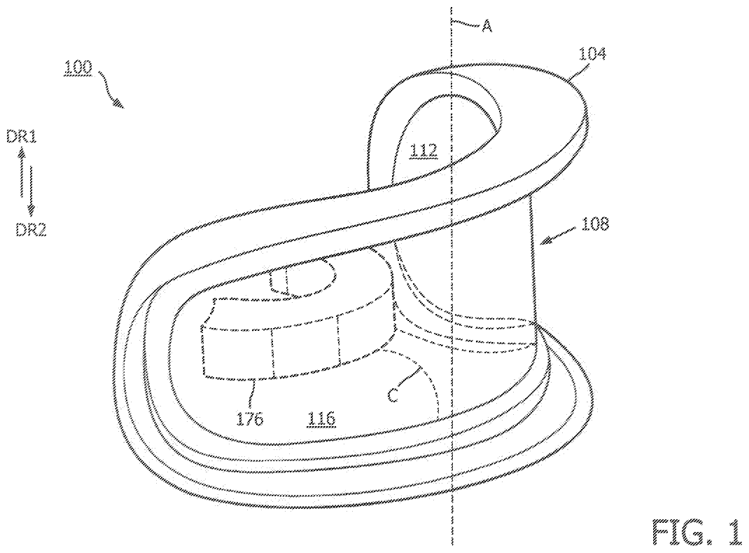

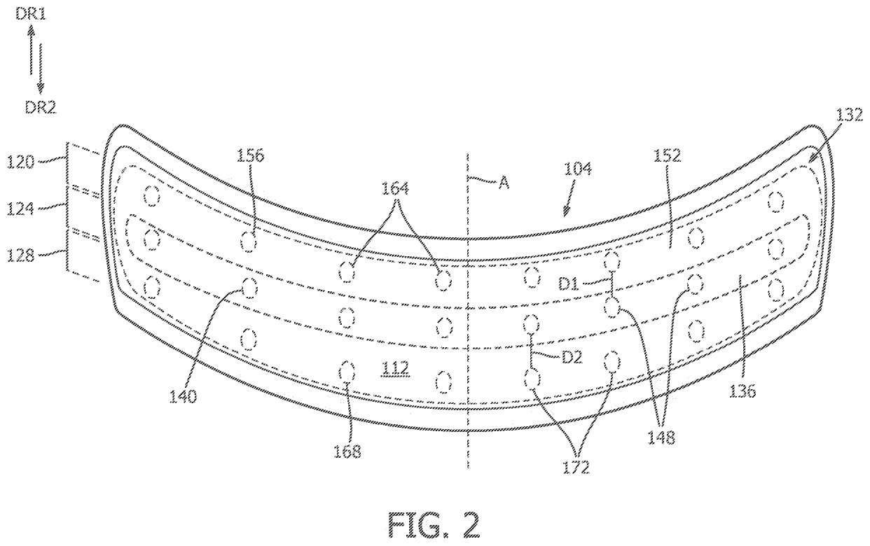

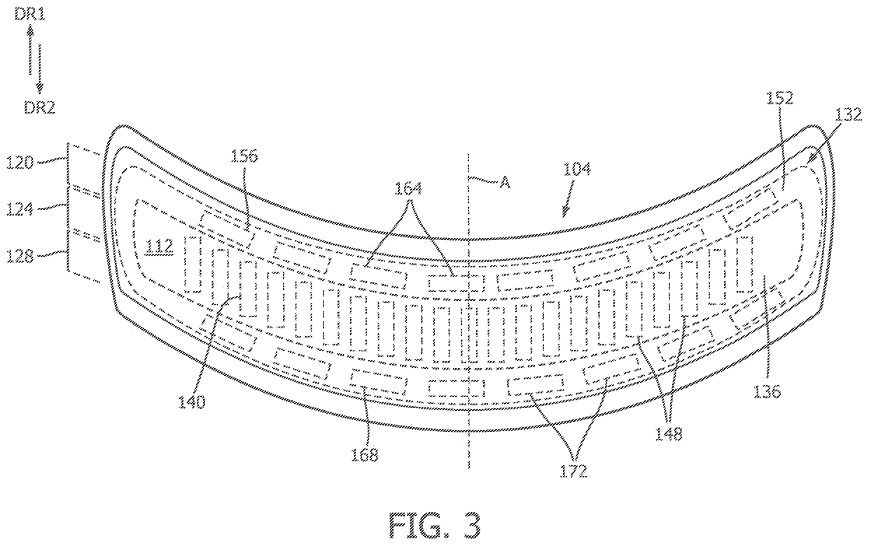

Referring to the figures, FIG. 1 illustrates a perspective view of oral care appliance 100 according to the present disclosure. Oral care appliance 100 has body 104. Body 104 includes plate member 108 which passes through an imaginary axis A, a lighting system 132 (shown in FIGS. 2 and 3), and a bite member 176. Axis A is substantially orthogonal to the surface of the ground beneath the user while using oral care appliance 100. Plate member 108 includes first surface 112 and second surface 116 substantially defining a curved plate. First surface 112 is intended to face a user's teeth T while they are engaged with bite member 176, while second surface 116 is intended to face away from the user's teeth T and away from the user when engaged with bite member 176. Plate member 108, which includes first surface 112 and second surface 116, is configured such that it forms an ergonomic, curved shape conforming substantially about a set of human teeth T. Plate member 108 can be made from materials such as, but not limited to, glass, plastics such as: Polyethylene Terephthalate, High Density Polyethylene (HDPE), Polyvinyl Chloride (PVC), Low-Density Polyethylene (LDPE), Polypropylene, Poly(methyl methacrylate)(PMMA), acrylic, or any material capable of being placed in a human mouth that at least partially reduces the attenuation of light through from first surface 112 to second surface 116 as explained in detail below. Alternatively, it should be appreciated that the material chosen for plate member 108 can be transparent or translucent.

FIG. 2 illustrates an embodiment of oral care appliance 100 and plate member 108.

First surface 112 of plate member 108 is, schematically, separated into three general portions, a top portion 120, a center portion 124, and a bottom portion 128. Plate member 108 is configured such that first surface 112 is substantially planar in a vertical dimension, while being substantially curved in a horizontal dimension with a substantially constant radius of curvature. As used herein, the term “vertical dimension” is used to describe a dimension of the first surface 112 that is substantially parallel with axis A, while the term “horizontal dimension” is used to describe a dimension of the first surface 112 that is substantially orthogonal to axis A. Top portion 120 is defined as the upper most portion of first surface 112 in first direction DR1 parallel to axis A. Bottom portion 128 is defined as the lower most portion of first surface 112 in first direction DR1 parallel to axis A. Center portion 124 is defined as the portion of first surface 112 located substantially between top portion 120 and bottom portion 128 along axis A.

Lighting system 132 includes at least two lighting sub-systems, i.e., first lighting sub-system 136 and second lighting sub-system 152. First lighting sub-system 136 includes at least one first light source 140. It should be appreciated that at least one first light source 140 may be one of a plurality of first light sources 148. At least one first light source 140 is arranged to produce a first light 142 at a first wavelength of light 144. First wavelength of light 144 is intended in to be within a range of electromagnetic radiation, generally within the range of 300-600 nm, specifically between 400-495 nm, which substantially corresponds with “blue” light; however, a different light wavelength can be used. Second lighting sub-system 152 includes at least one second light source 156. It should be appreciated that at least one second light source 156 may be one of a plurality of second light sources 164. At least one second light source 156 is arranged to produce a second light 162 at a second wavelength of light 160. Second wavelength of light 160 is intended to be within a range of electromagnetic radiation, generally within the range of 600-1500 nm, specifically between 650-1400 nm, which substantially corresponds with “red” or “Near Infrared” light. It can be appreciated that the second wavelength of light can with a different light wavelength than the red or infrared spectrum, depending on the desired outcome desired; however, first and second light wavelengths should differ.

Although, in a preferred embodiment, at least one first light source 140 and at least one second light source 156 are intended to be point-source light-emitting diodes (LEDs), it should be appreciated that at least one first light source 140 and at least one second light source 156 can be chosen from the group of: a light diffusing fiber (e.g., single mode, multimode, or photonic crystal fiber), an electrodeless light source, an incandescent light source, a florescent light source, a hollow-cathode light source, a sulfur light source, a neon and argon light source, a laser light source, and a plasma light source.

Furthermore, in some embodiments lighting system 132 includes at least one third light source 168. At least one third light source 168 can be a part of second lighting sub-system 152, or be a part of a separate third lighting sub-system (not shown). The at least one third light source 168 is arranged to produce the second light 162 at the second wavelength of light 160, i.e., light substantially in the “red” or “near infrared” spectrums. It should be appreciated that at least one third light source may be one of a plurality of third light sources 172.

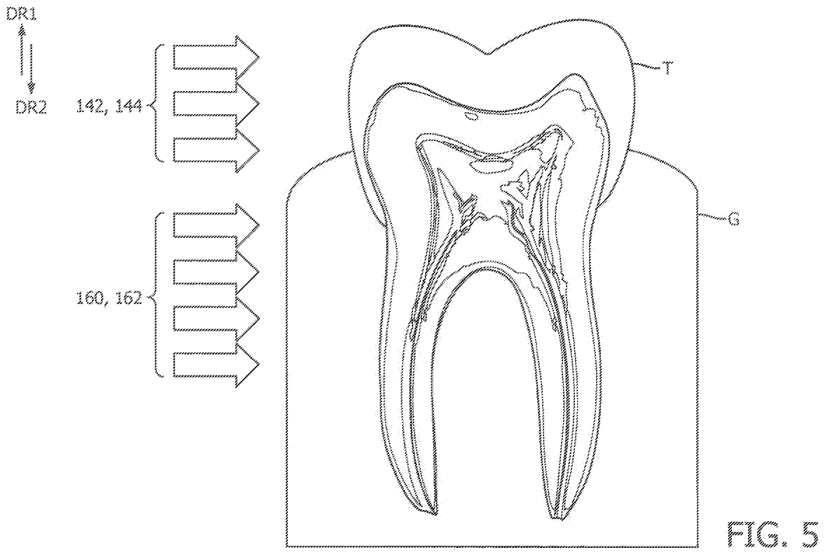

The at least one first light source 140 is separated from the at least one second light source 156 a first vertical distance D1 along axis A in direction DR1, such that a substantial quantity of the first wavelength of light 144 emitted by the at least one first light source 140 is directed to the user's teeth T (shown in FIG. 5), while a substantially quantity of the second wavelength of light 160 emitted by the at least one second light source 156 is directed to the user's gums G (shown in FIG. 5). Additionally, at least one third light source 168 is separated from at least one first light source 140 by a second vertical distance D2 along axis A, where vertical distance D2 is substantially equal to distance D1, in second direction DR2, opposite first direction DR1, such that a substantial quantity of the second wavelength of light 160 emitted by the at least one third light source 168 is directed to the user's gums G (shown in FIG. 5).

FIG. 3 illustrates an embodiment of oral care appliance 100 having plate member 108. Here, first surface 112 contains at least one first light source 140, at least one second light source 156, and at least one third light source 168 within center portion 124, top portion 120, and bottom portion 128, respectively, where the at least one first, second, and third light sources are arrays of LEDs instead of point-source LEDs shown in FIG. 2.

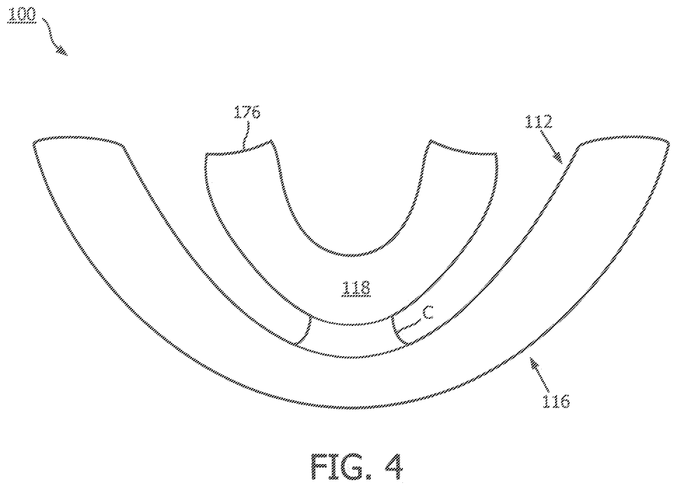

FIG. 4 illustrates a top plan view of the oral care appliance 100 shown in FIG. 1. In this view, bite member 176 and bite surface 180 can be seen. During use, the user places bite member within the user's mouth and provides a biting force on at least bite surface 180. In one example embodiment, bite member 176 is configured such that it substantially forms a negative of the user's mouth, similar to a various known mouth guards used for sporting events, with the exception of a front wall that would normally protect a user's teeth from contact. Without this front wall, bite member 176 allows for the light produced by at least one first light source 140, at least one second light source 156, and at least one third light source 168 to reach the user's teeth and gums without obstruction. Additionally, plate member 108 is configured such that during operation, the user's lips would encompass plate member 108 leaving a direct, unobstructed, path for the light produced by the at least one first light source 140, at least one second light source 156, and at least one third light source 168 to contact the user's teeth and gums. Bite member 176 is engaged with plate member 108 by a connector C. Although connector C is illustrated as being integral with plate member 108 and bite member 176, it should be appreciated that connector C could be removeably engaged with plate member 108.

FIG. 5 illustrates a cross-sectional view of a user's teeth T and gums G. In this view, the effect of the vertical separation of at least one first light source 140 and at least one second light source 156 can be seen. The separation these light sources first distance D1 creates a more efficient concentration of blue light directed at the user's teeth T, as well as a more efficient concentration of red or near infrared light directed at the user's gums G. It should be appreciated that although only one tooth and gum section is shown, this concept applies to the plurality of user's teeth T throughout the top and bottom rows of teeth within the user's mouth.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. An oral care appliance comprising:
a body, the body comprising:
a plate member having a first surface, the first surface comprising:
a top portion;
a center portion; and
a bottom portion; and
a lighting system comprising:
a first lighting sub-system having at least one first light source, the at least one first light source arranged in the center portion of the plate member;
a second lighting sub-system having at least one second light source, the at least one second light source arranged in the top portion of the plate member or the bottom portion of the plate member;
wherein the at least one first light source is arranged to produce a first light at a first wavelength in the range of 400-495 nm and the at least one second light source is arranged to produce a second light at a second wavelength in the range of 650-1400 nm.

2. The oral care appliance of claim 1, wherein the body further comprises a bite member having a bite surface arranged to contact a user's teeth.

3. The oral care appliance of claim 1, wherein the first surface of the plate member is a curved surface.

4. (canceled)

5. (canceled)

6. The oral care appliance of claim 1, wherein the at least one first light source is separated from the at least one second light source by a first vertical distance.

7. The oral care appliance of claim 1, wherein at least one of the at least one first light source and the at least one second light source is a light-emitting diode (LED).

8. The oral care appliance of claim 1, wherein the at least one first light source and the at least one second light source are selected from the group of: a light diffusing fiber, an electrodeless light source, an incandescent light source, a florescent light source, a hollow-cathode light source, a sulfur light source, a neon and argon light source, a laser light source, and a plasma light source.

9. The oral care appliance of claim 1
wherein the second lighting sub-system further comprises at least one third light source, the at least one second light source arranged in the top portion of the plate member and the at least one third light source arranged in the bottom portion of the plate member; and
wherein the at least one third light source is arranged to produce a second light at a second wavelength.

10. (canceled)

11. (canceled)

12. (canceled)

13. The oral care appliance of claim 9, wherein the at least one third light source and the at least one first light source are separated by a second vertical distance.

14. (canceled)

15. (canceled)
